(19) United States
(12) Patent Application Publication
(10) Pub. No.: US 2009/0324774 A1
(43) Pub. Date: Dec. 31, 2009

(54) METHOD FOR FEEDING MOTHER PIG

(75) Inventors: Akira Shiga, Miyazaki (JP); Akira Kamoshida, Kanagawa (JP); Yojiro Sasa, Niigata (JP); Shin Taniguchi, Niigata (JP); Kozo Nishibori, Niigata (JP)

Correspondence Address: LUCAS & MERCANTI, LLP, 475 PARK AVENUE SOUTH, 15TH FLOOR, NEW YORK, NY, 10016, US

(73) Assignee: YUKIGUNI MAITAKE CO., LTD., Minamiuonuma-shi, Niigata (JP)

(21) Appl. No.: 12/520,936

(22) PCT Filed: Mar. 15, 2007
(86) PCT No.: PCT/JP2007/055180

§ 371 Date: Jul. 20, 2009

(30) Foreign Application Priority Data
Dec. 28, 2006 (JP) 2006-354443

(51) Int. Cl. A23K 1/18; A61K 36/07

(52) U.S. Cl. 426/2

(57) ABSTRACT

An object of the present invention is to maintain good conditions of mother pigs and decrease the stillborn piglets and the death rate of piglets in breast-feeding period, so as to increase the productivity in pig farming.

In a method for feeding pigs, by administering a Grifola-derived substance selected from one or more of dried Grifola, dried Grifola powder and a Grifola extract to mother pigs, particularly to postpartum mother pigs, good conditions of mother pigs can be maintained and the stillborn piglets and the death rate of piglets in breast-feeding period can decrease. As a result, the productivity in pig farming can increase.

### TECHNICAL FIELD

The present invention relates to a method for feeding pigs using dried Grifola, dried Grifola powder and a Grifola extract. More specifically, the present invention relates to a method for feeding mother pigs, which maintains good conditions of mother pigs and decreases the number of stillborn piglets and the death rate of piglets in breast-feeding period by administering the “Maitake” mushroom (Grifola frondosa) to mother pigs, so as to increase the productivity in pig farming.

### BACKGROUND ART

In pig-farming industry, the productivity of domestic pigs has seriously decreased due to diseases. As competition in producing domestic animals increases, priority has been put on economical and efficient production. As a result, the comfortable environment for feeding pigs has not been provided. Therefore, various diseases can easily occur in pigs raised under stressful conditions caused by the change in feed technology and the deterioration of the feeding environment. A spate of the pig diseases reduces the farm productivity and gives pig farmers big economic losses. Consequently, it is important to prevent diseases by enhancing the autoimmunity of pigs, in order to increase the productivity in pig farming.

Particularly, the health care of mother pigs greatly influences the productivity. It is considered that improving conditions of mother pigs leads to an increase in the lactation yield in lactation period and gives a great effect to prevent diarrhea in piglets not only in breast-feeding period but also in postweaning period. Additionally, the deterioration of conditions of mother pigs in lactation period leads to a delay in return of estrus and a decrease in the number of piglets to-be-born, and greatly affects the productivity in pig farming.

Since the conditions of mother pigs greatly affect the productivity in pig-farming industry, various studies for improving the productivity in pig farming by maintaining good conditions of mother pigs have been made.

Particularly, feeds and feed additives, which can control conditions of mother pigs, decrease the developing rate of diarrhea and mortality of piglets, and which can increase the number of born piglets, and methods for feeding pigs using the feeds have been proposed. A feed containing galacto-oligosaccharide [JP Patent Publication 1]; a casein digest-containing feed mainly comprising casein phosphopeptide [JP Patent Publication 2]; a method for providing a bacterial cell and a disrupted bacterial cell [JP Patent Publication 3]; a feed containing a guar gum enzymatic decomposition product [JP Patent Publication 4]; a feed using a reduced (activated) folic acid and a method for feeding the same [JP Patent Publications 5, JP Patent Publication 6]; a feed for feeding female pigs for breeding containing sugar mainly comprising oligosaccharide [JP Patent Publication 7]; a method for administering a feed mixed with an electrolyte-controlled mineral [JP Patent Publication 8]; a technique characterized by administering transferrin to pregnant mother pigs [JP Patent Publication 9]; and a technique for administering an yeast extract containing ribonucleic acid or 5′-nucleotide [JP Patent Publication 10] have been disclosed.

A technique for feeding a mixed feed for mother pigs has been proposed, the mixed feed being controlled to have 25 to 35% by weight of methionine, 65 to 75% by weight of threonine, 15 to 21% by weight of triptophan, and 83 to 93% by weight of valine, with respect to lysine; containing 10000 to 12000 IU/kg of vitamin A, 1500 to 2500 IU/kg of vitamin D3, 40 to 100 mg/kg of vitamin E and 1 to 3 mg/kg of vitamin K3; or being controlled to have 3200 to 3400 kcal/kg of digestible energy [JP Patent Publication 11].

Additionally, an agent for preventing bacterial infection for animals, by which born piglets can be prevented from diarrhea and skin infection during lactation period when used to treat antepartum mother pigs, has been proposed [JP Patent Publication 12]. A feed additive utilizing excellent properties of Schizophyllum commune culture which is a natural product, and a feed additive characterized by containing a finely ground object of the Schizophyllum commune culture and an yeast have been proposed [JP Patent Publication 13].

However, a technique for increasing the productivity in pig farming by utilizing the “Maitake” mushroom (Grifola frondosa) which has been reported as having an immunostimulating property to humans, particularly by applying the same to mother pigs, and for controlling the conditions of mother pigs has not been proposed.

The inventors of the present application have studied about components contained in the “Maitake” mushroom (Grifola frondosa) and the use of the same, and found the immunostimulating property of Grifola for humans, so as to discover various effects. For example, the inventors have discovered that a fraction obtained by purifying a Grifola hydrothermal extract by an alcohol treatment has an immunostimulating property and an antitumor effect [JP Patent Publication 14] and that the fraction usefully effects in influenza virus defense against infection [JP Patent Publication 15], and have filed patent applications.

The inventors of the present application have studied also about application of the “Maitake” mushroom (Grifola frondosa) to domestic livestock feeding and have filed a patent application regarding an additive for a livestock feed containing a Grifola-derived substance [JP Patent Publication 16]. However, the invention relates to a feed additive for domestic animals, which enables production of meat with high safety and good flavor, and is not particularly intended to maintain good conditions of mother pigs and to decrease the stillborn piglets and the death rate of piglets in breast-feeding period, so as to increase the productivity in pig farming.
JP Patent Publication 1: (Kokai) No. 5-219897 A
JP Patent Publication 2: (Kokai) No. 5-268883 A
JP Patent Publication 3: (Kokai) No. 5-336896 A
JP Patent Publication 4: (Kokai) No. 7-8183 A
JP Patent Publication 5: (Kokai) No. 7-147911 A
JP Patent Publication 6: (Kokai)No. 8-70788 A
JP Patent Publication 7: (Kokai) No. 8-23889 A
JP Patent Publication 8: (Kokai) No. 10-304825 A
JP Patent Publication 9: (Kokai) No. 11-4637 A
JP Patent Publication 10:(Kokai) No. 2004-313178 A
JP Patent Publication 11: (Kokai) No. 2005-192514 A
JP Patent Publication 12: (Kokai) No. 8-59474 A
JP Patent Publication 13: (Kokai) No. 7-170919 A
JP Patent Publication 14: (Kokai) No. 9-238697 A
JP Patent Publication 15: (Kokai) No. 2005-145934 A
JP Patent Publication 16: (Kokai) No. 2003-259816 A

### DISCLOSURE OF THE INVENTION

### Problems to be Solved by the Invention

An object of the present invention is to maintain good conditions of mother pigs and decrease the stillborn piglets and the death rate of piglets in breast-feeding period, so as to increase the productivity in pig farming.

### Means for Solving the Problems

The inventors have focused on the “Maitake” mushroom (Grifola frondosa) reported as having an immunostimulating property and administered dried Grifola powder to mother pigs to determine the effect of dried Grifola to pigs raising. The inventors discovered that the number of stillborn piglets and the number of dead pigs in breast-feeding period are decreased by administering the powder, and thus have completed the present invention.

That is, the present invention relates to:

(1) a method for feeding pigs, wherein a Grifola-derived substance selected from one or more of dried Grifola, dried Grifola powder and a Grifola extract is administered to mother pigs; and
(2) the method for feeding pigs described in (1), wherein mother pigs are postpartum mother pigs.

In the present invention, all types of Grifola including “Maitake” (Grifola frondosa), “Shiromaitake” (Grifola albicans), “Choreimaitake” (Dendropolyporus umbellatus), “Tonbimaitake” (Grifola gigantea) and the like can be used. Recently, artificial cultivation of the fruit bodies of “Maitake” (Grifola frondosa) has been made possible. It is preferable to use the fruit bodies of Grifola in terms of ensuring a stable supply of raw materials.

Fresh Grifola, dried Grifola or dried Grifola powder can be used. The dried powder is easy to handle and preferable. As dried Grifola, any Grifola prepared by sun drying, hot-air drying or freeze drying can be used.

Dried Grifola can be used as it is, or can also be used in the form of small chips, small pieces or fine pieces after appropriate grinding. It is more general to grind dried Grifola into powder using a milling apparatus or the like for use, because this leads to a broader application range. In addition, depending on the feeding situation, Grifola powder can be appropriately selected from those with large particle sizes to Grifola finer powder with small particle sizes.

Dried Grifola, dried Grifola powder or a Grifola extract can be added independently to a feed. To enable thorough mixing of the Grifola added to the feed, dried Grifola, dried Grifola powder or a Grifola extract in the dosage form of powders mixed with an extender and a lubricant, granules, pellets, or the like prepared by general methods can also be added to the feed. When a case of using Grifola in the form of powder is explained as an example, an extender and a lubricant are added to the dried Grifola powder, and then the resultant is thoroughly mixed. At this time, if necessary, substances other than a Grifola-derived substance can be added.

As an extender, lactose, starch or dextrin can be used, for example. As a lubricant, a light liquid paraffin can be used. Particularly, when a Grifola extract is used, it is preferable to mix the Grifola extract with an extender because the Grifola extract becomes easily moistened.

Additionally, a medical agent for preventing pig diseases can be used together with dried Grifola, dried Grifola powder or a Grifola extract. As a medical agent, a drug for preventing pneumonia or a disease in digestive system can be fed. Also, a microorganism material including lactobacillus having an immunostimulating effect can be used.

It can be assumed that the method for administering dried Grifola according to the present invention to domestic pigs raising improves immunostimulating ability of the pigs. Therefore, the feed can be administered to not only antepartum and postpartum mother pigs but also pigs at any feeding stage with no problem. The method for feeding dried Grifola powder according to the present invention provides effects when administered to pigs for a certain period. The feeding period varies depending on the kind of the pig or the feeding stage. However, feeding pigs in the period from 7 days prior to delivery of piglets to weaning of piglets can be a rough idea. It is preferable to give the feed continuously during the period. The feeding amount is preferably around 0.06 g to 0.11 g/kg/day.

In the present invention, “mother pigs” represents copulating pigs to pigs weaning piglets, preferably pigs in the period from one week prior to delivery of piglets to weaning of piglets.

### Effects of the Invention

By administering a Grifola-derived substance selected from one or more of dried Grifola, dried Grifola powder and a Grifola extract to mother pigs, good conditions of mother pigs can be maintained and the stillborn piglets and the death rate of piglets in breast-feeding period can decrease, so as to increase the productivity in pig farming.

This specification includes the contents as disclosed in the description and/or drawings of Japanese Patent Application No. 2006-354443, which is a priority document of the present application.

### BEST MODE FOR CARRYING OUT THE INVENTION

The present invention is described in detail below using Example. However, the present invention is not limited by the Example.

### EXAMPLE 1

Production of Dried Grifola, Dried Grifola Powder and Grifola Extract

(1) Artificially cultivated fruit bodies of fresh Grifola frondosa were lined on the racks of a drying room with racks, and then dried by blowing hot air at approximately 60° C. to approximately 80° C. By raising the temperature stepwise initially from 60° C. to finally 80° C. for almost one day so as to perform heating and drying, dried fruit bodies of Grifola frondosa were obtained. Subsequently, the dried Grifola frondosa was powdered with a milling apparatus.

On the other hand, a Grifola frondosa extract can be produced with water, alcohol, etc., according to general methods.

(2) At the HS farm, from May to June of 2005, the dried Grifola frondosa powder obtained according to the above-described method was fed every day at the ratio of 0.5% by weight with respect to the weight of the feed, to 80 mother pigs in the period from 7 days prior to delivery of piglets to weaning of piglets.

Next, the number of born piglets from the mother pigs, the number of piglets starting suckling, the number of stillborn piglets, the body weight, the suckling rate, the length of gestation, the number of weaned piglets and the weaning rate were checked every month. Table 1 represents the reference group, and Table 2 represents the example group.

From the results shown in Tables 1 and 2, the average number of stillborn piglets in the example group is 0.56 in comparison with that of the reference group, 1.29. Therefore, it can be seen that the number of stillborn piglets decreased by half in the present invention. That is, it can be recognized that the number of stillborn piglets is decreased by administering dried Grifola powder. Additionally, it can be said that the productivity in pig farming increases as the death rate of piglets in breast-feeding period decreases.

The suckling rate, the weaning rate and the final yield of the example group are higher than those of the reference group.

TABLE 1

| | | | | | The | | | | | | | | | | |
|---|---|---|---|---|---|---|---|---|---|---|---|---|---|---|---|
| | | | | The | number of | | | | | | | | | | |
| | | No. of | | number | piglets | | | | | | | Foster | | Body | |
| | Delivery | mother | Birth | of born | starting | Stillborn piglets | | | | | | piglets | | weight | Suckling |
| | date | pig | history | piglets | suckling | Frail | Lentigo | Albino | Deformation | Others | Total | + | − | at birth | rate |
| 1 | May, 24 | 6064 | 1 | 13 | 12 | 0 | 0 | 1 | 0 | 0 | 1 | 0 | 0 | 1.4 | 95.3 |
| 2 | May, 27 | 5667 | 8 | 11 | 10 | 0 | 0 | 0 | 1 | 0 | 1 | 0 | 0 | 1.4 | 90.9 |
| 3 | May, 27 | 5669 | 7 | 11 | 10 | 0 | 1 | 0 | 0 | 0 | 1 | 0 | 0 | 1.5 | 90.9 |
| 4 | June, 16 | 6026 | 4 | 14 | 11 | 0 | 1 | 2 | 0 | 0 | 3 | 0 | 0 | 1.4 | 78.5 |
| 5 | June, 24 | 5668 | 8 | 15 | 14 | 0 | 0 | 1 | 0 | 0 | 1 | 0 | 1 | 1.2 | 93.3 |
| 6 | June, 24 | 6018 | 6 | 12 | 10 | 0 | 0 | 2 | 0 | 0 | 2 | 0 | 0 | 1.3 | 83.3 |
| 7 | June, 24 | 6054 | 2 | 10 | 10 | 0 | 0 | 0 | 0 | 0 | 0 | 1 | 0 | 1.4 | 100 |

| | | | | | | | | | | | | | |
|---|---|---|---|---|---|---|---|---|---|---|---|---|---|
| Average | 5.1 | 12.3 | 11.0 | 0.0 | 0.3 | 0.9 | 0.1 | 0.0 | 1.29 | 0.1 | 0.1 | 1.37 | 89.89 |

| | | | | The | | | | | | | | |
|---|---|---|---|---|---|---|---|---|---|---|---|---|
| | | No. | | number of | | Breast- | | | | Dead piglets in breast-feeding | | |
| | Delivery | of mother | Length of | weaned | Weaning | feeding | Final | Body weight | Body weight | period | | |
| | date | pig | gestation | piglets | rate | period | yield | when weaned | at 21 days old | Crush | Diarrhea | Debilitation |
| 1 | May, 24 | 6064 | 116 | 9 | 75 | 27 | 69 | 7.0 | 5.72 | 1 | 0 | 0 |
| 2 | May, 27 | 5667 | 115 | 10 | 100 | 24 | 91 | 7.5 | 6.75 | 0 | 0 | 0 |
| 3 | May, 27 | 5669 | 115 | 8 | 80 | 24 | 73 | 7.1 | 6.39 | 1 | 1 | 0 |
| 4 | June, 16 | 6026 | 118 | 11 | 100 | 22 | 79 | 5.8 | 5.59 | 0 | 0 | 0 |
| 5 | June, 24 | 5668 | 112 | 10 | 79 | 27 | 73 | 7.1 | 5.80 | 3 | 0 | 0 |
| 6 | June, 24 | 6018 | 112 | 9 | 90 | 27 | 75 | 6.8 | 5.56 | 1 | 0 | 0 |
| 7 | June, 24 | 6054 | 112 | 10 | 90 | 27 | 90 | 6.8 | 5.56 | 1 | 0 | 0 |

| | | | | | | | | | | |
|---|---|---|---|---|---|---|---|---|---|---|
| Average | 114.3 | 9.6 | 87.64 | 25.4 | 78.51 | 6.87 | 5.91 | 1.0 | 0.1 | 0.0 |

TABLE 2

| | | | | | The | | | | | | | | | | |
|---|---|---|---|---|---|---|---|---|---|---|---|---|---|---|---|
| | | | | The | number of | | | | | | | | | | |
| | | No. of | | number | piglets | | | | | | | Foster | | Body | |
| | Delivery | mother | Birth | of born | starting | Stillborn piglets | | | | | | piglets | | weight | Suckling |
| | date | pig | history | piglets | suckling | Frail | Lentigo | Albino | Deformation | Others | Total | + | − | at birth | rate |
| 1 | May, 26 | 6065 | 1 | 11 | 10 | 0 | 1 | 0 | 0 | 0 | 1 | 0 | 0 | 1.6 | 90.9 |
| 2 | May, 30 | 6066 | 1 | 8 | 7 | 0 | 0 | 1 | 0 | 0 | 1 | 0 | 0 | 1.4 | 87.5 |
| 3 | June, 2 | 6009 | 6 | 12 | 11 | 0 | 0 | 1 | 0 | 0 | 1 | 0 | 0 | 1.3 | 91.6 |
| 4 | June, 2 | 5670 | 8 | 13 | 13 | 0 | 0 | 0 | 0 | 0 | 0 | 0 | 0 | 1.4 | 100 |
| 5 | June, 2 | 5580 | 7 | 13 | 12 | 0 | 1 | 0 | 0 | 0 | 1 | 0 | 0 | 1.0 | 92.3 |
| 6 | June, 2 | 5530 | 10 | 13 | 12 | 0 | 1 | 0 | 0 | 0 | 1 | 0 | 0 | 1.1 | 92.3 |
| 7 | June, 16 | 5639 | 8 | 12 | 12 | 0 | 0 | 0 | 0 | 0 | 0 | 0 | 0 | 1.3 | 100 |
| 8 | June, 13 | 6025 | 4 | 17 | 17 | 0 | 0 | 0 | 0 | 0 | 0 | 0 | 0 | 1.0 | 100 |
| 9 | June, 22 | 6045 | 3 | 14 | 14 | 0 | 0 | 0 | 0 | 0 | 0 | 0 | 0 | 1.5 | 100 |

| | | | | | | | | | | | | | |
|---|---|---|---|---|---|---|---|---|---|---|---|---|---|
| Average | 5.3 | 12.6 | 12.0 | 0.0 | 0.3 | 0.2 | 0.0 | 0.0 | 0.56 | 0.0 | 0.0 | 1.29 | 94.96 |

| | | | | The | | | | | | | | |
|---|---|---|---|---|---|---|---|---|---|---|---|---|
| | | No. | | number of | | Breast- | | | | Dead piglets in breast-feeding | | |
| | Delivery | of mother | Length of | weaned | Weaning | feeding | Final | Body weight | Body weight | period | | |
| | date | pig | gestation | piglets | rate | period | yield | when weaned | at 21 days old | Crush | Diarrhea | Debilitation |
| 1 | May, 26 | 6065 | 114 | 10 | 100 | 25 | 91 | 7.3 | 6.35 | 0 | 0 | 0 |
| 2 | May, 30 | 6066 | 112 | 7 | 100 | 30 | 88 | 9.0 | 6.75 | 0 | 0 | 0 |
| 3 | June, 2 | 6009 | 114 | 11 | 100 | 27 | 92 | 6.8 | 5.56 | 0 | 0 | 0 |
| 4 | June, 2 | 5670 | 114 | 11 | 84.6 | 27 | 85 | 6.8 | 5.56 | 1 | 0 | 0 |
| 5 | June, 2 | 5580 | 114 | 12 | 100 | 27 | 92 | 5.5 | 4.50 | 0 | 0 | 0 |
| 6 | June, 2 | 5530 | 114 | 12 | 100 | 27 | 92 | 5.5 | 4.50 | 0 | 0 | 0 |
| 7 | June, 16 | 5639 | 115 | 10 | 83.3 | 26 | 83 | 6.4 | 5.40 | 2 | 0 | 0 |
| 8 | June, 13 | 6025 | 114 | 15 | 88.2 | 29 | 88 | 6.4 | 4.93 | 1 | 0 | 1 |
| 9 | June, 22 | 6045 | 118 | 12 | 85.7 | 29 | 86 | 6.5 | 5.01 | 2 | 0 | 0 |

| | | | | | | | | | | |
|---|---|---|---|---|---|---|---|---|---|---|
| Average | 114.3 | 11.1 | 93.53 | 27.4 | 88.49 | 6.69 | 5.40 | 0.7 | 0.0 | 0.1 |

* Suckling rate = the number of piglets starting suckling/the number of born piglets
Weaning rate = the number of weaned piglets/the number of piglets starting suckling

All publications, patents and patent applications cited herein are incorporated herein by reference in their entirety.

1. A method for feeding pigs, comprising:
administering one or more of Grifola-derived substance selected from the group consisting of dried Grifola, dried Grifola powder and a Grifola extract to mother pigs.

2. The method according to claim 1, wherein the mother pig is a postpartum mother pig.
